(19) United States
(12) Patent Application Publication
(10) Pub. No.: US 2008/0293721 A1
(43) Pub. Date: Nov. 27, 2008

(54) ARYLTHIOACETAMIDE CARBOXYLATE DERIVATIVES AS FKBP INHIBITORS FOR THE TREATMENT OF NEUROLOGICAL DISEASES

(75) Inventors: Maurizio Pellecchia, San Diego, CA (US); Jinhua Chen, San Diego, CA (US); Ziming Zhang, San Diego, CA (US)

Correspondence Address: DLA PIPER US LLP, 4365 EXECUTIVE DRIVE, SUITE 1100, SAN DIEGO, CA, 92121-2133, US

(73) Assignee: Burnham Institute for Medical Research, La Jolla, CA (US)

(21) Appl. No.: 12/112,991

(22) Filed: Apr. 30, 2008

Related U.S. Application Data
(60) Provisional application No. 60/927,601, filed on May 4, 2007.

(51) Int. Cl. C07D 401/12; C07D 265/30; A61K 31/5375; A61K 31/5377; A61P 27/00; A61P 25/00; A61P 31/00

(52) U.S. Cl. 514/237.2; 544/158; 514/237.5; 544/131

(57) ABSTRACT

The present invention provides compounds having the general structure I, or a pharmaceutically acceptable salt thereof:

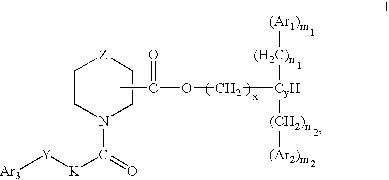

wherein Z is selected from a group consisting of O, C, N, and S; each of Ar₁, Ar₂, and Ar₃ is a moiety selected from a group consisting of an aryl and a substituted aryl; Y is selected from a group consisting of S, O, CH₂ and SO₂; K is selected from a group consisting CH₂, CF₂, O, NH, and SO₂; each of n₁ and n₂ is an integer selected from a group consisting of 0, 1, 2, 3, and 4; and each of x, y, m₁ and m₂ is an integer selected from a group consisting of 0 and 1. Pharmaceutical compositions based on compounds I are also provided for treatment of a neurological disorders, diseases, or pathologies, and for treatment of infectious diseases.

### CROSS REFERENCE TO RELATED APPLICATIONS

This application claims the benefit of priority under 35 U.S.C. § 119(e) of U.S. Ser. No. 60/927,601 filed May 4, 2007, the entire content of which is incorporated herein by reference.

### GOVERNMENT FUNDING

The invention described herein was made with government support under Grant Number 1 X01 MH078942-01, awarded by the National Institute of Health. The United States Government has certain rights in the invention.

### BACKGROUND

1. Field of the Invention

The invention relates generally to compounds useful for the treatment of neurological disorders, diseases, and pathologies, as well as for the treatment of infectious diseases. More specifically, it relates to inhibitors of FK506 binding proteins (FKBP) and to the use thereof.

2. Background Information

Previously, some inhibitors of FKBP have been identified, including rapamycin and tacrolimus. However, such products bind to various portions of FKBP, including those unrelated to the treatment of neurological disorders, thus reducing the potency of the inhibitor. Other products that have been studied include certain ketoamides. While such ketoamides can inhibit FKBP, it is difficult to use them due to the fact that these compounds include metabolically unstable α-ketoamide portion.

### SUMMARY

Currently, there is a need for novel, potent, and selective agents for the treatment of neurological or disorders, diseases, and pathologies, as well as for the pharmaceutical compositions including such agents. Such agents can be based on inhibitors of FK506 binding proteins (FKBP), such as inhibitors of the protein FKBP12.

According to one embodiment of the present invention, compounds are provided having the general structure I:

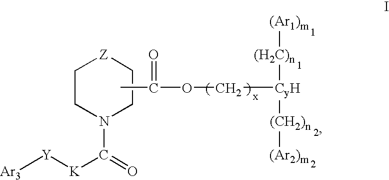

wherein Z is selected from a group consisting of O, C, N, and S; each of Ar₁, Ar₂, and Ar₃ is a moiety selected from a group consisting of an aryl and a substituted aryl; Y is selected from a group consisting of S, O, CH₂ and SO₂; K is selected from a group consisting CH₂, CF₂, O, NH, and SO₂; each of n₁ and n₂ is an integer selected from a group consisting of 0, 1, 2, 3, and 4; and each of x, y, m₁ and m₂ is an integer selected from a group consisting of 0 and 1.

According to other embodiments of the present invention, pharmaceutical compositions are provided for the treatment of neurological disorders, diseases, and pathologies, the compositions comprising a compound having the general structure I and a pharmaceutically acceptable carrier.

According to other embodiments of the present invention, pharmaceutical compositions are provided for the treatment of infectious diseases, the compositions comprising a compound having the general structure I and a pharmaceutically acceptable carrier.

According to other embodiments of the present invention, methods for the treatment of neurological disorders, diseases, and pathologies, and methods for the treatment of infectious diseases are provided, the methods comprising administering to a subject in need thereof a pharmacologically effective dose of a pharmaceutical composition comprising a compound having the general structure I.

### BRIEF DESCRIPTION OF THE DRAWINGS

FIG. 1 shows the data on binding of a specific ligand to FKBP12, according to an embodiment of the invention.

FIG. 2 shows secondary assays with isothermal titration calorimetry and 2D NMR where kD=4 μM, according to an embodiment of the invention.

FIG. 3 shows secondary assays with isothermal titration calorimetry and 2D NMR where kD=9 μM, according to an embodiment of the invention.

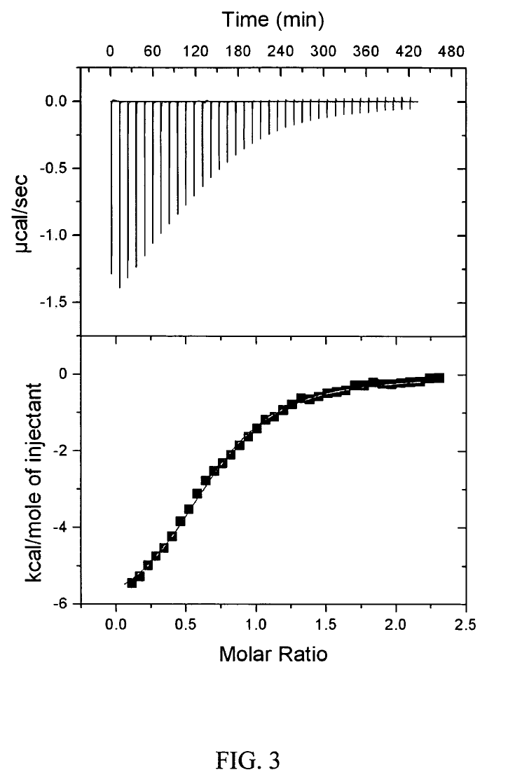

FIG. 4 shows the effect of FKBP12 ligands on neurite outgrow with primary rat cortical neurons, according to an embodiment of the invention where the ligand is 3-(pyridine-3-yl)propyl 4-(2-(4-isopropylphenylthio)acetyl)morpholine-3-carboxylate.

FIG. 5 shows the Sholl analysis demonstrating the effect of FKBP12 ligands on neurite outgrow with primary rat cortical neurons, according to an embodiment of the invention.

### DETAILED DESCRIPTION

The following definitions are used, unless otherwise described.

Alkyl, alkoxy, alkenyl, alkynyl, etc. denote both straight and branched groups; but reference to an individual radical such as “propyl” embraces only the straight chain radical, while a branched chain isomer such as “isopropyl” is referred to specifically.

The term “aryl” denotes a phenyl radical or an ortho-fused bicyclic carbocyclic radical having about nine to ten ring atoms in which at least one ring is aromatic. The term “aryl” for the purposes of the present invention is inclusive of “heteroaryl,” which encompasses a radical attached via a ring carbon of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto. An example of heteroaryl is pyridyl.

As used herein, the term “FKBP” refers to an isomerase, which is a protein of immunophilin group that catalyzes peptidyl-propyl cis-trans isomerization and has affinity to FK506 drugs and antibiotics. On example of a typical FKBP protein is the protein FKBP12.

The term “FK506” (also known as “tacrolimus”) refers to a chemical compound which is a 23-membered macrolide lactone, having the Chemical Abstracts Reference 104987-11-3.

As used herein, the term “patient” refers to organisms to be treated by the methods of the present invention. Such organisms include, but are not limited to, humans. In the context of the invention, the term “subject” generally refers to an individual who will receive or who has received treatment for the treatment of a disease, disorder or pathology

According to embodiments of the present invention, compounds are provided having the general structure I, or pharmaceutically acceptable salts thereof:

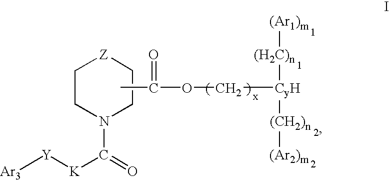

wherein Z is selected from a group consisting of O, C, N, and S; each of Ar₁, Ar₂, and Ar₃ is a moiety selected from a group consisting of an aryl and a substituted aryl; Y is selected from a group consisting of S, O, CH₂ and SO₂; K is selected from a group consisting CH₂, CF₂, O, NH, and SO₂; each of n₁ and n₂ is an integer selected from a group consisting of 0, 1, 2, 3, and 4; and each of x, y, m₁ and m₂ is an integer selected from a group consisting of 0 and 1.

Examples of some sub-genera that are within the purview of the present invention and are described by the general structure I include the structures where Y is S, and K is CH₂, i.e., the structures II, III, and IV:

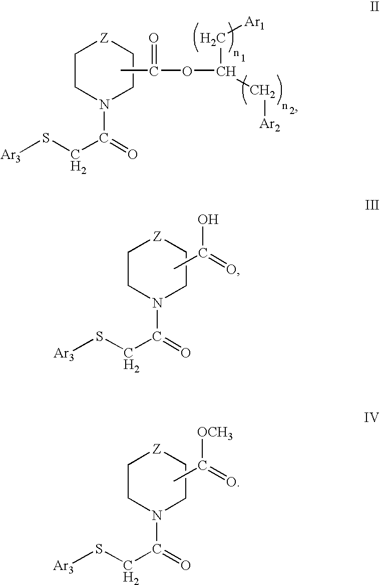

More narrowly, examples of some sub-genera that are within the purview of the present invention and are described by the general structure I include the structures where Z is 0, Ar₃ is p-isopropylphenyl (i.e., is derived from cumene), and Ar₁ is m-pyridyl.

Some non-limiting examples of specific compounds that are within the purview of the present invention and are described by the general structure I, include the compounds described by the formulae V, VI, and VII:

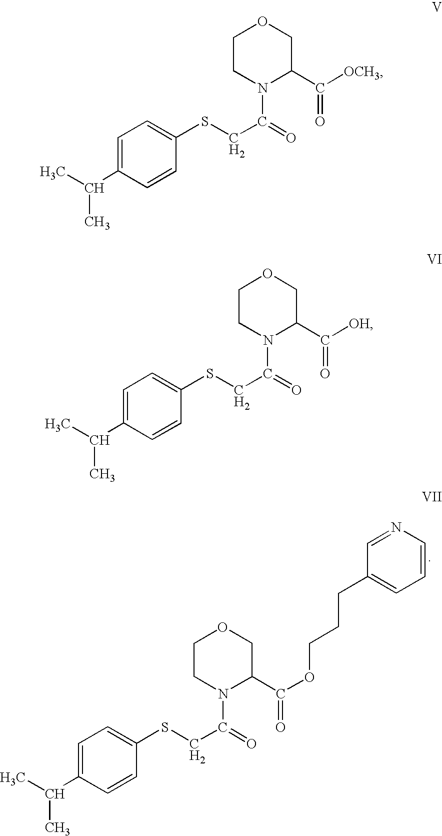

The compounds of the present invention, when brought in contact with a FKBP protein such as FKBP12, inhibit such a protein and accordingly serve as agents for the treatment of both neurological disorders, diseases, and pathologies, and of infectious diseases. Accordingly, the compounds having the structure I or pharmaceutically acceptable salts thereof can be used for preparing pharmaceutical compositions, e.g., by combining these compounds and pharmaceutically acceptable carriers. The pharmaceutical compositions can then be used in pharmacologically effective doses for the treatment of a neurological disorder, disease, or pathology. Non-limiting examples of the neurological disorders, diseases, or pathologies that can be so treated include neurodegeneration, nerve injury, and vision and memory disorders. In addition, the pharmaceutical compositions based on the compounds having the structure I can be also used in pharmacologically effective doses for the treatment of infectious diseases.

Various synthetic schemes can be designed for manufacturing the products having the general structure I. One such scheme is shown as scheme (A) below and the synthesis can be outlined as follows.

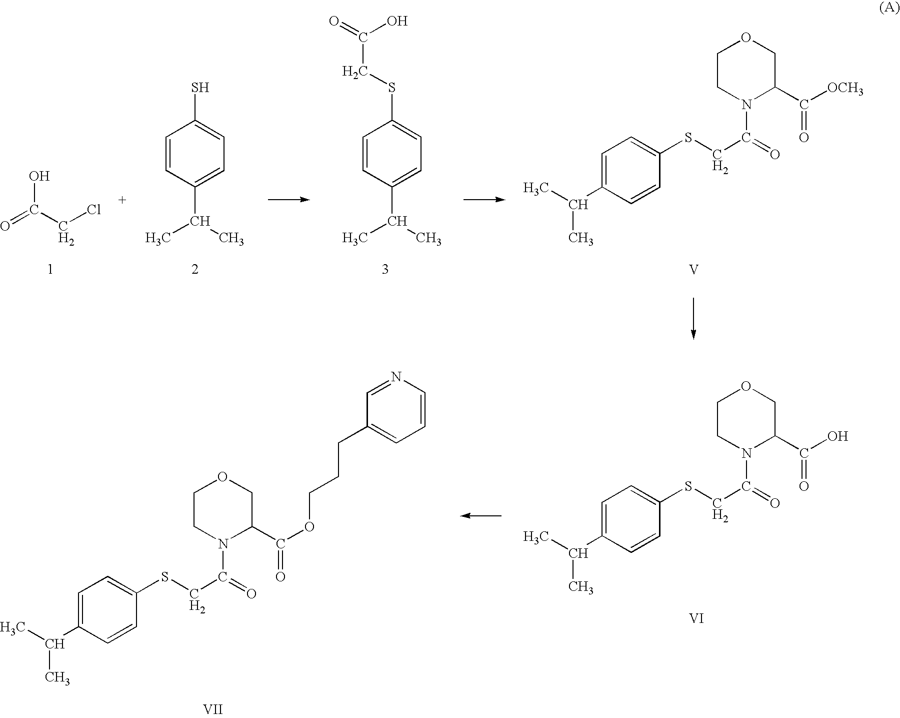

As shown by the reaction scheme (A), the synthesis starts by combining 2-chloroacetic acid 1 with 4-isopropylbenzenethiol 2, followed by adding aqueous sodium hydroxide. The reaction typically lasts about 2 hours at 80° C., followed by adding hydrochloric acid, to obtain the intermediate 3, i.e., 2-(4-isopropylbenzenethio)acetic acid.

The intermediate 3 can then be treated with N-ethyl-N′-(3-dimethylaminopropyl)-carbodiimide (EDC) and (1-hydroxy-1H-benzotriazole hydrate) (HOBt), followed by adding triethylamine and methyl morpholine-3-carboxylate in dimethylformamide (DMF). The reaction is carried out for about 12 hours at room temperature to afford methyl 4-(2-(4-isopropylphenylthio)acetyl)morpholine-3-carboxylate (compound V).

Compound V can then be treated with lithium hydroxide, in aqueous tetrahydrofuran (THF), for about 5 hours at room temperature, followed by adding hydrochloric acid, to afford 4-(2-(4-isopropylphenylthio)acetyl)morpholine-3-carboxylic acid (compound VI).

Finally, compound VI can be then converted into compound VII by reacting compound VI with EDC, dimethylaminopyridine (DMAP), and 3-(pyridin-3-yl)propan-1-ol, in the DMF solution. The reaction is carried out for about 12 hours at room temperature to afford 3-(pyridine-3-yl)propyl 4-(2-(4-isopropylphenylthio)acetyl)morpholine-3-carboxylate (compound VII).

Pharmaceutically acceptable salts of the compounds of the present invention may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The above-described compounds I-VII can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds I-VII can be determined by comparing their in vitro activity, and in vivo activity in animal models. Methods for the extrapolation of effective dosages in mice, and other animals, to humans are known to those having ordinary skill in the art who can, for example, be guided by the procedures described in U.S. Pat. No. 4,938,949.

Generally, the concentration of the compound(s) I-VII in a liquid composition, such as a lotion, can be between about 0.1 and 25 mass %, such as between about 0.5 and 10 mass %. The concentration in a semi-solid or solid composition such as a gel or a powder can be between about 0.1 and 25 mass %, such as between about 0.5 and 2.5 mass %.

The amount of the compound(s) I-VII, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose can be in the range of between about 0.5 and 100 mg/kg, e.g., between about 10 and 75 mg/kg of body weight per day, such as between about 15 and 60 mg/kg/day. The compound(s) I-VII can be conveniently administered in unit dosage form; for example, containing 5 to 1000 mg, such as 10 to 750 mg, for example, 50 to 500 mg of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The following example are intended to further illustrate but not limit the invention.

### EXAMPLE 1

### Synthesis of 2-(4-isopropylphenylthio)acetic acid (BI-69A4) (Intermediate 3)

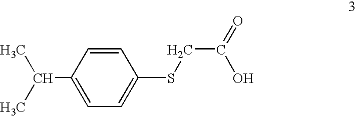

4-isopropylbenethiol (762 mg, 5.0 mmol) (i.e., the intermediate 2 on the reaction scheme (A) above) was dissolved in a NaOH solution (500 mg, 12.5 mmol) in H₂O (1.2 mL). 2-chloroacetic acid (472.5 mg, 5.0 mmol) (i.e., the intermediate 1 on the reaction scheme (A) above) was added and the reaction mixture was heated at 80° C. for 2 hours under nitrogen. The reaction mixture was dissolved in H₂O (100 mL) and was adjusted to pH=2.0 with concentrated HCl, followed by extraction with ethyl acetate (EtOAc). The combined organic phase was extracted with Na₂CO₃ solution (10%). The Na₂CO₃ solution was then adjusted to pH 2.0 with concentrated HCl and extracted with EtOAc. It was dried over Na₂SO₄ and solvent was removed to afford the title intermediate 3 as a white solid, which was later used without further purification. Yield, 450 mg (43%).

¹H NMR (300 MHz, DMSO-d6) δ 7.28-7.18 (m, 4H), 3.73 (s, 2H), 2.84 (sept, J=6.9 Hz, 1H), 1.17 (d, J=6.9 Hz, 6H). ¹³C NMR (75 MHz, CDCl₃) δ 176.1, 148.6, 131.0, 127.5, 37.3, 33.9, 24.0. HRMS m/z calculated for C₁₁H₁₃O₂S [M-H]⁻ 209.0642, found 209.0635.

### EXAMPLE 2

### Synthesis of methyl 4-(2-(4-isopropylphenylthio)acetyl)morpholine-3-carboxylate (Compound V)

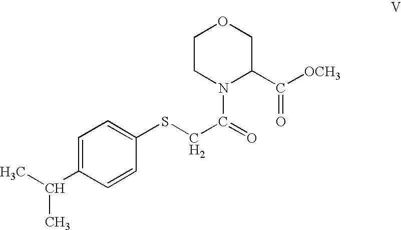

Triethylamine (150 mg, 1.5 mmol), EDC (114.6 mg, 0.6 mmol), and HOBt (91.8 mg, 0.6 mmol) were added to a solution of the intermediate 3 described above (105 mg, 0.5 mmol) in DMF (5 mL) under N₂. After the reaction mixture was stirred at room temperature for 30 minutes, methyl morpholine-3-carboxylate (90.5 mg, 0.5 mmol) was added, followed by further stirring at room temperature for 12 hours and by removal of DMF under reduced pressure. The final product was purified by silica gel flash chromatography (EtOAc-Hexanes=1:4) to afford the title compound V as a colorless oil. Yield, 70 mg (42%).

The title compound V exists in two amide rotamers in about 10:1 ratio. For the major rotamer: ¹H NMR (300 MHz, CDCl₃): δ 7.44-7.35 (m, 2H), 7.21-7.14 (m, 2H), 5.02 (s, 1H), 4.41 (d, J=12 Hz, 1H), 3.94-3.79 (m, 2H), 3.76 (s, 3H), 3.73 (s, 1H), 3.64-3.45 (m, 3H), 3.48-3.36 (m, 1H), 2.94-2.82 (m, 1H), 1.26-1.18 (m, 6H). ¹³C NMR (75 MHz, CDCl₃): δ 169.7, 169.1, 148.5, 131.4, 127.3, 67.7, 66.3, 52.6, 52.5, 43.8, 36.9, 33.8, 23.9. HRMS m/z calculated for C₁₇H₂₄NO₄S [M+H]⁺ 338.1426, found 338.1423.

### EXAMPLE 3

### Synthesis of 4-(2-(4-isopropylphenylthio)acetyl)morpholine-3-carboxylic acid (Compound VI)

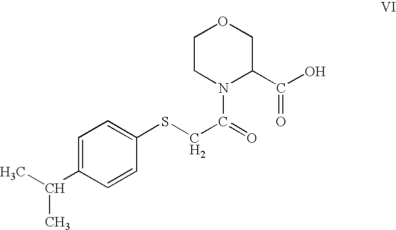

A solution of the above-described compound V (337 mg, 1.0 mmol) in THF (10 mL) was mixed with a solution of lithium hydroxide (120 mg, 5.0 mmol) in H₂O (10 mL). After the mixture was stirred at room temperature for 5 hours, THF was removed by reduced pressure and the solution was washed with ether (3×20 mL). The solution was then adjusted to pH=1.0 with concentrated HCl and was extracted with ether. The combined ether solution was dried over Na₂SO₄. The solvent was removed to afford the title compound VI without further purification. Yield, 200 mg (62%).

¹H NMR (300 MHz, CDCl₃): δ 7.62 (b, 1H), 7.42-7.36 (m, 2H), 7.20-7.14 (m, 2H), 5.04 (d, J=3.3 Hz, 1H), 4.43 (d, J=11.7, 1H), 3.94-3.70 (m, 3H), 3.70-3.50 (m, 3H), 3.50-3.34 (m, 1H), 2.87 (sept, J=6.9 Hz), 1.23 (s, 3H), 1.21 (s, 3H). ¹³C NMR (75 MHz, CDCl₃): δ 173.5, 169.7, 148.7, 131.7, 127.3, 67.5, 66.1, 52.4, 43.8, 36.8, 33.7, 23.8. HRMS m/z calculated for C₁₆H₂₂NO₄S [M+H]⁺ 324.1264, found 324.1263.

### EXAMPLE 4

### Synthesis of 3-(pyridine-3-yl)propyl 4-(2-(4-isopropylphenylthio)acetyl)morpholine-3-carboxylate (Compound VII)

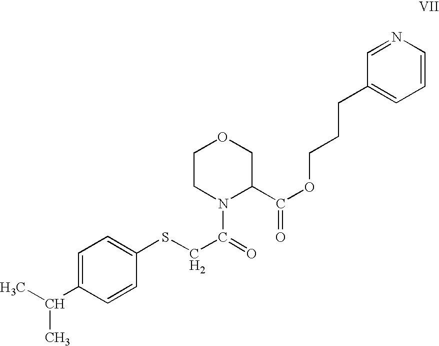

3-(pyridin-3-yl)propan-1-ol (56 mg, 0.41 mmol) was added to a solution of the above-described compound VI (110 mg, 0.34 mmol) in DMF (5 mL). EDC (95 mg, 0.51 mmol) and DMAP (20 mg, 0.17 mmol) were added. After the reaction mixture was stirred at room temperature for 12 hours, DMF was removed under reduced pressure and the residue was purified by silica gel flash chromatography (EtOAc-Hexanes=1:2-1:1) to afford the title compound VII as a white solid. Yield, 80 mg (53%).

¹H NMR (300 MHz, CDCl₃): δ 8.46-8.42 (m, 2H), 7.50-7.45 (m, 1H), 7.42-7.34 (m, 2H), 7.23-7.19 (m, 1H), 7.19-7.13 (m, 2H), 5.00 (d, J=3.6 Hz, 1H), 4.44-4.34 (m, 1H), 4.25-4.16 (m, 2H), 3.94-3.82 (m, 1H), 3.78-3.42 (m, 1H), 3.70-3.54 (m, 3H), 3.48-3.38 (m, 1H), 2.86 (sept, J=6.9 Hz, 1H), 2.67 (t, J=7.2 Hz, 2H), 2.04-1.92 (m, 2H), 2.21 (d, J=6.9 Hz, 6 H). ¹³C NMR (75 MHz, CDCl₃): δ 169.3, 150.0, 147.7, 136.0, 131.4, 127.4, 123.5, 67.9, 66.4, 64.7, 52.7, 43.9, 37.0, 33.8, 30.0, 29.3, 24.0. HRMS m/z calculated for C₂₄H₃₁N₂O₄S [M+H]⁺ 443.1999, found 443.2007.

### EXAMPLE 5

### Binding Information

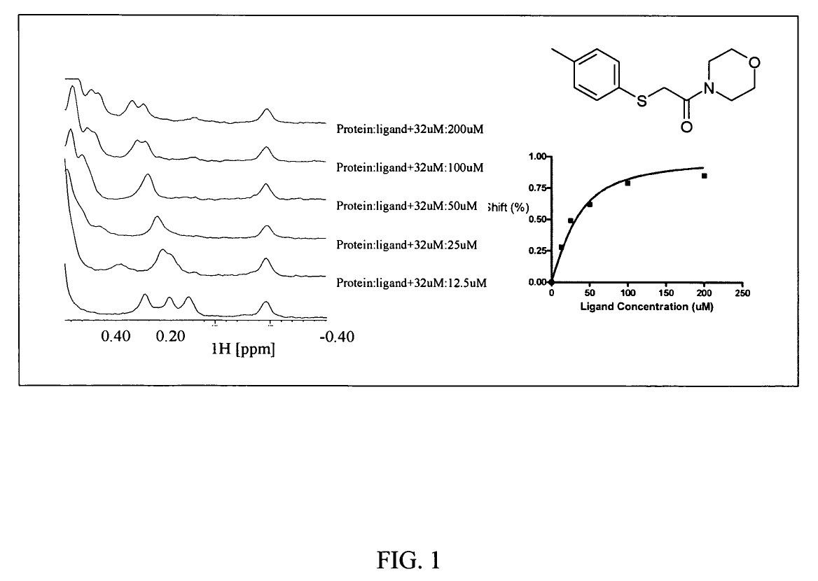

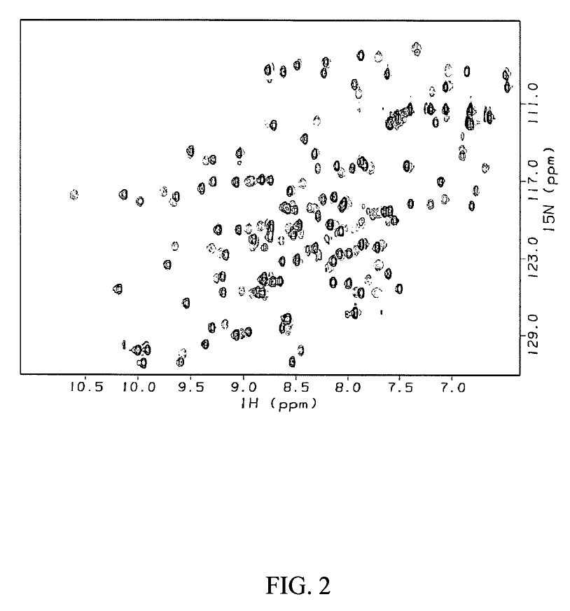

FIG. 1 demonstrates the data on binding of a specific ligand to FKBP12. To obtain these data, about 4,000 compounds were screened by NMR, and the hits were followed with dose response curves for Kd determination. The ligand concentration chart refers to the compound having the formula VIII (i.e., p-toluoylthioacetylmorpholine) used as the ligand. FIG. 2 demonstrates secondary assays with isothermal titration calorimetry and 2D NMR for the same ligand.

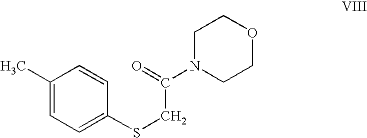

As shown by FIGS. 1 and 2, the compound VIII has good affinity to FKBP12. As can be seen from the comparison of the structures VI and VIII, and VII, both compounds VI and VII share common structural features with compound VIII, including the presence of both the morpholine moiety and thioacetyl moiety. Accordingly, it can be concluded that compounds VI and VII have sub-micromolar affinity for FKBP12.

### EXAMPLE 6

### Neurite Growth

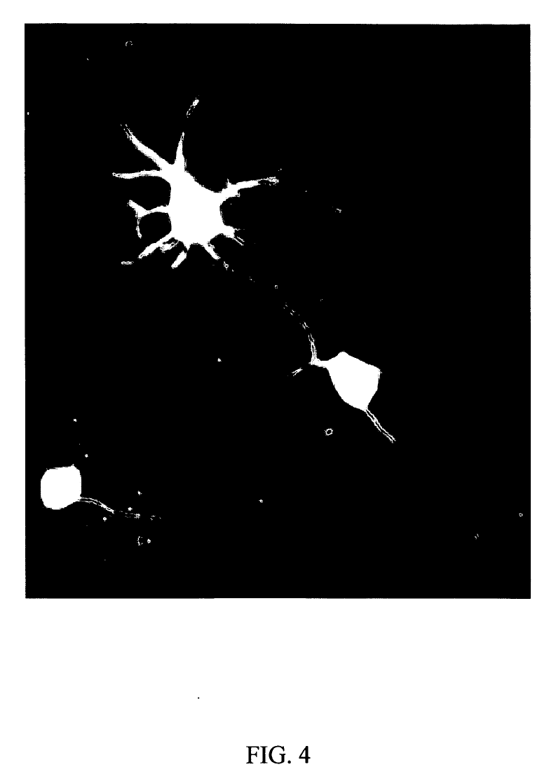

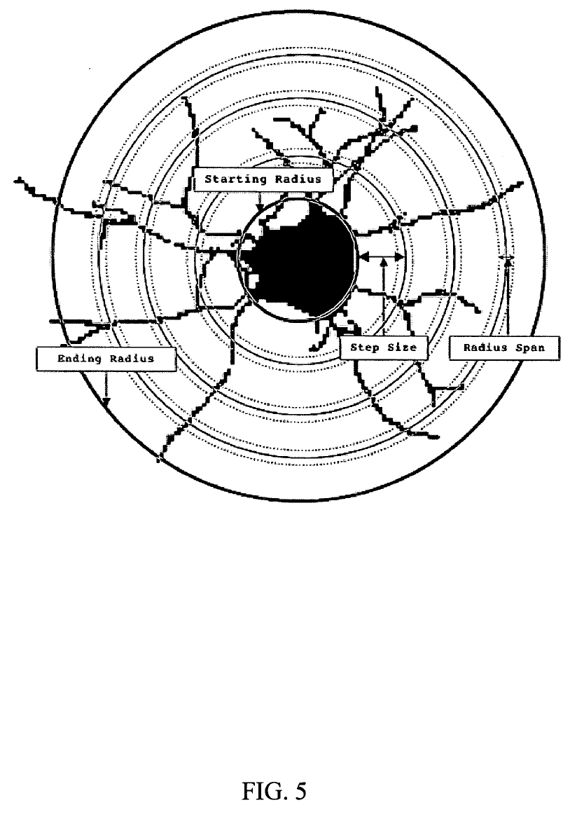

FIGS. 4 and 5 show the effect of FKBP12 ligands on neurite outgrow with primary rat cortical neurons. As can be seen the effect is particularly substantial where the ligand is the compound VII (FIG. 4).

Although the invention has been described with reference to the above example, it will be understood that modifications and variations are encompassed within the spirit and scope of the invention. Accordingly, the invention is limited only by the following claims.

What is claimed is:

1. A compound having the general structure (I), or a pharmaceutically acceptable salt thereof: 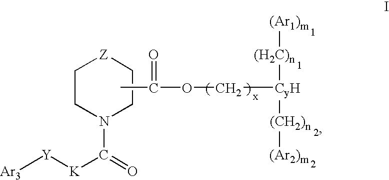
wherein:
Z is selected from a group consisting of O, C, N, and S;
each of Ar₁, Ar₂, and Ar₃ is a moiety selected from a group consisting of an aryl and a substituted aryl;
Y is selected from a group consisting of S, O, CH₂ and SO₂;
K is selected from a group consisting CH₂, CF₂, O, NH, and SO₂;
each of n₁ and n₂ is an integer selected from a group consisting of 0, 1, 2, 3, and 4; and
each of x, y, m₁ and m₂ is an integer selected from a group consisting of 0 and 1.

2. The compound of claim 1, wherein the general structure of the compound is selected from a group consisting of structures II, III, and IV:

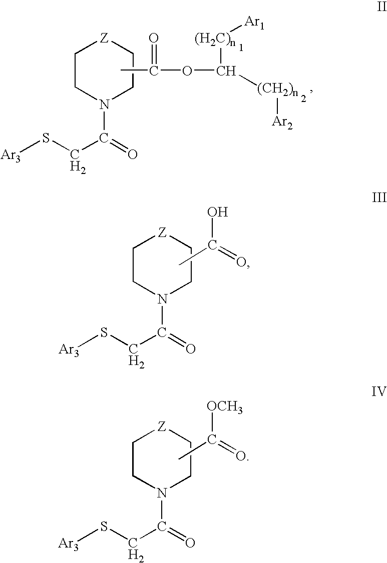

3. The compound of claim 1 or 2, wherein Z is O.

4. The compound of claim 1, wherein Ar₃ is p-isopropylphenyl.

5. The compound of claim 1, wherein Ar₁ is m-pyridyl.

6. The compound of claim 1, wherein the compound is selected from a group consisting of structures V, VI, VII, and VII:

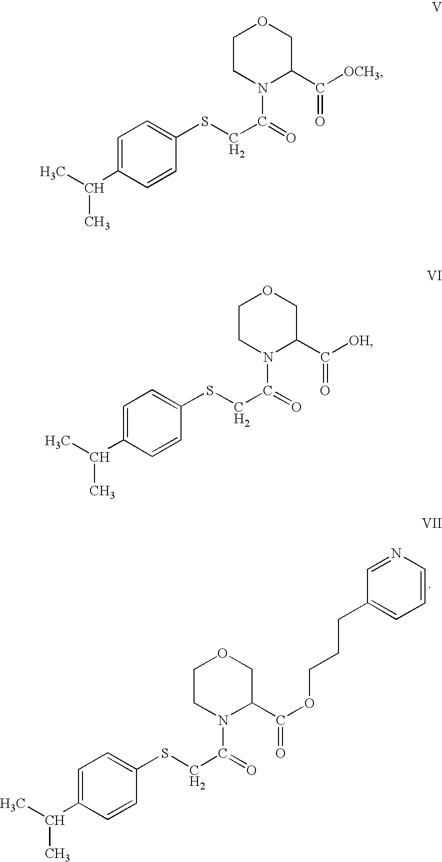

7. A pharmaceutical composition comprising a compound of claim 1 and a pharmaceutically acceptable carrier therefor.

8. A method of treatment of a neurological disorder, disease, or pathology, comprising administering a pharmacologically effective dose of a pharmaceutical composition of claim 7 to a subject in need thereof, thereby treating the neurological disorder, disease, or pathology.

9. The method of claim 8, wherein the neurological disorder, disease, or pathology is selected from a group consisting of neurodegeneration, a nerve injury, a vision disorder and a memory disorder.

10. A method of treatment of an infectious disease comprising administering a pharmacologically effective dose of the pharmaceutical composition to a subject in need thereof, thereby treating the infectious disease.
